(19) United States
(12) Patent Application Publication
(10) Pub. No.: US 2003/0059955 A1
(43) Pub. Date: Mar. 27, 2003

(54) Affinity tag modified particles

(76) Inventor: Cynthia C. Bamdad, Newton, MA (US)

Correspondence Address: Timothy J. Oyer, Wolf, Greenfield & Sacks, P.C., 600 Atlantic Avenue, Boston, MA, 02210, US

(21) Appl. No.: 10/233,334

(22) Filed: Aug. 30, 2002

Related U.S. Application Data
(60) Provisional application No. 60/316,510, filed on Aug. 31, 2001.

(51) Int. Cl. G01N 33/551

(52) U.S. Cl. 436/524

(57) ABSTRACT

The present invention provides methods, assays, kits, and components for the detection and analysis of binding between various biological or chemical species, as well as techniques for facilitating the attachment of various biological or chemical species to a particle. In some cases, particles having the ability to emit electromagnetic radiation within a narrow wavelength band, for example, semiconductor nanocrystals, are attached to a substrate or a structure, such as a molecule, a particle, a fluid sample, a cell, or a tissue. The attachment may be a direct attachment or an indirect attachment, for example, an attachment comprising an affinity tag/recognition entity interaction. The particles may then be further used to assay biological or chemical entities, or combined with other detection techniques.

### BACKGROUND

1. Field of the Invention

This invention generally relates to chemical and biochemical detection methods and, more particularly, to techniques for facilitating the attachment of a species with a particle having the ability to emit electromagnetic radiation.

2. Description of the Related Art

Chemical, biological, and biochemical screening and assay techniques for determining binding interactions between various species are well known. However, genuinely high-throughput techniques are relatively rare. Instead, typical techniques involve laborious, sequential tests involving a variety of binding candidates to determine binding interactions. Generally required, for true high-throughput screening techniques, are simultaneous detection of differential signals in a one-step assay, and convenient techniques for immobilizing chemical, biological, or biochemical species to components of these assays.

A semiconductor nanocrystal is a particle that comprises a semiconductor material and is typically about 1-100 nm in diameter. The wavelength at which the semiconductor nanocrystal fluoresces may depend on the size of the nanocrystal, and the emission wavelength may be controlled by controlling the particle diameter. The emission profile of a semiconductor nanocrystal may be very narrow and highly symmetric, and may not directly depend on the excitation wavelength. For example, one excitation wavelength may be used to excite a population of semiconductor nanocrystals having different sizes, resulting in the emission of many different wavelengths of light due to the excitation wavelength. This property may be used to simultaneously resolve multiple semiconductor nanocrystals present within a given sample (e.g., “multiplexing”). Semiconductor nanocrystals have previously been described in, for example, U.S. Pat. No. 6,274,323 by Bruchez et al., U.S. Pat. No. 6,207,392 by Weiss, et al., or U.S. Pat. No. 5,990,479 by Weiss, et al.

Semiconductor nanocrystals have been suggested for use as biological probes. The use of semiconductor nanocrystals can allow multiple tests for multiple targets to be performed within the same sample, allowing simultaneous rather than sequential detection. However, using existing techniques (such as chemical coupling) to attach biological probes such as proteins to nanocrystals can present certain difficulties. For example, one method for attaching proteins to surfaces is to react a carboxylate on the surface with an exposed primary amine on the protein using standard EDC/NHS coupling chemistry. However, when used with particles, rather than planar surfaces, this approach can be problematic, because proteins usually have several exposed primary amines; this may lead to one protein being coupled to several particles, resulting in occlusion of binding sites or precipitation of the particles. Additionally, chemical coupling may denature the protein in some cases, thus compromising the assay. Further, chemical attachment of probe proteins to the carrier particle may be inconvenient or time-consuming, and generally requires expertise often not possessed by the end user.

Although various techniques for detecting chemical, biological, and biochemical interactions are known, improved techniques that potentially can be more sensitive, can discriminate between various binding interactions, and can lead to higher throughput are needed. It also would be of significant value to increase versatility in attachment of chemical, biological, and biochemical entities to components of screening and diagnostic assays.

### SUMMARY OF THE INVENTION

This invention relates to chemical and biochemical detection methods and, more particularly, to techniques for facilitating the immobilization of a species with respect to a particle having the ability to emit electromagnetic radiation. One significant aspect involves simplified, versatile techniques for linking chemical or biological (including, by definition, biochemical) entities to semiconductor nanocrystals via affinity tag/recognition entity pairs.

The subject matter of this application involves, in some cases, interrelated products, alternative solutions to a particular problem, and/or a plurality of different uses of a single system or article.

In one aspect, the invention includes a complex. In one set of embodiments, the complex includes a species able to emit electromagnetic radiation in a narrow wavelength band, and a member of an affinity tag/recognition entity pair immobilized relative to the species. In another set of embodiments, the complex includes an article, and a species able to emit electromagnetic radiation in a narrow wavelength band immobilized relative to the article via at least two binding partner pair interactions in series. In yet another set of embodiments, the complex includes an article comprising a particle comprising a first binding partner and a second binding partner not identical to the first binding partner, and a species able to emit electromagnetic radiation in a narrow wavelength band immobilized relative to the article.

In another aspect, the invention includes a system. The system, in one embodiment, includes an article, a first particle immobilized relative to the article via a first binding partner pair interaction, and a second particle immobilized relative to the article via a second binding partner pair interaction. The first particle includes a first species able to emit electromagnetic radiation in a first narrow wavelength band, and the second particle includes a second species able to emit electromagnetic radiation in a second narrow wavelength band.

The invention includes a method, in another aspect. In one embodiment, the method includes the steps of providing a complex comprising a particle and a chemical or biological entity, and exposing the complex to a fluid suspected of containing a substance able to bind to the chemical or biological entity. The complex is able to emit electromagnetic radiation in a narrow wavelength band. In another embodiment, the method includes the steps of providing a complex having a least two binding partner pair interactions in series, and exposing the complex to a fluid suspected of containing a substance able to bind to at least one of the at least two binding partner pair interactions. The complex is able to emit electromagnetic radiation in a narrow wavelength band.

The method, in yet another embodiment, includes the steps of providing a complex able to become immobilized relative to an article comprising at least a first binding partner and a second binding partner not identical to the first binding partner, and exposing the complex to a fluid suspected of containing a substance able to alter the ability of the complex to become immobilized relative to the article. The complex is able to emit electromagnetic radiation in a narrow wavelength band. The invention, in still another embodiment, includes a method of allowing a first particle to become immobilized relative to an article via a first binding partner pair interaction, and allowing a second particle to become immobilized relative to the article via a second binding partner pair interaction. The first particle includes a first species able to emit electromagnetic radiation in a first narrow wavelength band, and the second particle includes a second species able to emit electromagnetic radiation in a second narrow wavelength band.

In another aspect, the invention is directed to a method of making any of the embodiments described herein. In yet another aspect, the invention is directed to a method of using any of the embodiments described herein. In still another aspect, the invention includes a kit that can include, or can be used to produce any of the embodiments described herein.

Other advantages, novel features, and objects of the invention will become apparent from the following detailed description of non-limiting embodiments of the invention when considered in conjunction with the accompanying drawings, which are schematic and which are not intended to be drawn to scale. In the figures, each identical or nearly identical component that is illustrated in various figures typically is represented by a single numeral. For purposes of clarity, not every component is labeled in every figure, nor is every component of each embodiment of the invention shown where illustration is not necessary to allow those of ordinary skill in the art to understand the invention. In cases where the present specification and a document incorporated by reference include conflicting disclosure, the present specification shall control.

### BRIEF DESCRIPTION OF THE DRAWINGS

Non-limiting embodiments of the present invention will be described by way of example with reference to the accompanying drawings in which:

FIG. 1 is a schematic diagram of prior art;

FIG. 2 is a schematic diagram of one embodiment of the invention, showing two binding partner pair interactions in series;

FIG. 3 is a schematic diagram of one embodiment of the invention, showing a particle able to emit electromagnetic radiation in a narrow wavelength band immobilized to another particle; and

FIG. 4 is a schematic diagram of one embodiment of the invention, showing two particles able to emit electromagnetic radiation in a narrow wavelength band immobilized to another particle;

FIG. 5 is a schematic diagram of one embodiment of the invention, showing a series of particles on a surface;

FIG. 6 is a schematic diagram of one embodiment of the invention, showing a series of particles to be detected; and

FIG. 7 is a schematic diagram of one embodiment of the invention, showing a series of particles near an electrode.

### DETAILED DESCRIPTION

International patent application serial number PCT/US01/12484, filed Apr. 12, 2001 by Bamdad et al., entitled “Treatment of Neurodegenerative Disease” (International patent publication WO 01/78709, published Oct. 25, 2001), International patent application serial number PCT/US00/01997, filed Jan. 25, 2000 by Bamdad et al., entitled “Rapid and Sensitive Detection of Aberrant Protein Aggregation in Neurodegenerative Diseases” (International patent publication WO 00/43791, published Jul. 27, 2000), and International patent application serial number PCT/US00/01504, filed Jan. 21, 2000 by Bamdad, et al., entitled “interaction of Colloid-Immobilized Species with Species on Non-Colloidal Structures” (International patent publication WO 00/34783, published Jul. 27, 2000), all are incorporated herein by reference.

The present invention provides methods, assays, kits, and components for the detection and analysis of binding between various biological or chemical species, as well as techniques for facilitating the attachment of various biological or chemical species to a particle. In some cases, particles having the ability to emit electromagnetic radiation within a narrow wavelength band are attached to a substrate or a structure, such as a molecule, a particle, a fluid sample, a cell, or a tissue. The attachment may be a direct attachment or an indirect attachment, for example, an attachment comprising an affinity tag/recognition entity interaction. The particles may then be further used to assay biological or chemical entities, or combined with other detection techniques.

Major aspects of the present invention include, but are not limited to, the following. Tools for diagnostic, biomolecular studies, proteomic studies, or drug screening, including protein chips and particles for signaling interactions are contemplated. Multi-particle systems, such as two-particle systems, are also included. In multi-particle systems, one particle may be a recruitable particle and the other particle may carry a binding partner of an agent presented by the recruitable particle. In some cases, the particle may be a signaling entity; e.g., the particle may be able to emit energy such as electromagnetic energy in a narrow wavelength band, or the particle may carry or comprise an auxiliary signaling entity. Another major area in which the invention finds use involves cellular or biochemical studies, especially techniques involving studies of the interactions between ligands and cell surface proteins and receptors. In another aspect, the invention may be used for target identification, for example, in drug discovery, biochemical, or biomolecular studies. Another area in which the invention finds use involves the detection of analytes, such as proteins, hormones, or small molecules, either in solution or on the surfaces of intact cells, for example, for diagnostic purposes. As one example, the use of a semiconductor nanoparticle may be used in a diagnostic assay, where a variety of specimens, for example, from tissue or bodily fluids, may be probed for the presence of one or more targets.

Affinity tag/recognition entity pair interactions were developed to aid in protein purification and immobilization. Proteins may be modified at the genetic level with certain peptide sequences, known as affinity tags, that bind to known entities. Affinity tags generally fall into three categories: a) peptide sequences that bind to small molecules; b) fusion proteins that bind to small molecules; and c) peptide tags or fusion proteins that bind to antibodies. An affinity tag may also be a small molecule that has a convenient binding partner. The affinity tag may be covalently attached to a target protein, peptide, antibody, nucleic acid, or the like. In this way, affinity-tagged proteins and peptides may be immobilized on a particle that bears a binding partner, or a recognition entity, of the affinity tag. For example, nitrilo tri-acetic acid, when complexed to Ni²⁺ (NTA-Ni²⁺), defines a recognition entity that binds proteins modified with a stretch of histidines, known as a histidine tag, defining an affinity tag. NTA moieties can be readily coupled to a variety of molecules. For example, thiols terminated with NTA readily incorporate into self-assembled monolayers to form surface coatings that selectively capture histidine-tagged proteins and peptides.

Thus, in one set of embodiments of the invention, proteins or other species may be attached to small particles that emit in narrow wavelength range, such as semiconductor nanocrystal particles, either directly or indirectly, through the use of binding partner pair interactions such as recognition entity/affinity tag interactions. The species-particle complex may be used to facilitate the capture and presentation of affinity-tagged biomolecules. In one embodiment, detergent-like molecules or polymers may be derivatized with NTA-Ni²⁺ moieties and used to coat particles that emit electromagnetic radiation in a narrow wavelength band, such as semiconductor nanocrystals, to facilitate the attachment of histidine-tagged species, which can include, for example histidine-tagged DNA or proteinaceous species. As one example, molecules or polymers that bear glutathione or other small, detectable molecules may be adhered to the particle surface to capture glutathione-S-transferase (GST) fusion proteins. In some embodiments, a variety of antibodies may be conveniently presented by the particles, for example, if the particles are first coated with molecules or polymers that present Protein A, Protein G, Protein L, or fragments thereof. In a similar manner, proteins such as Proteins A, G, L, or fragments thereof may be histidine-tagged, then immobilized on nanoparticles bearing NTA-Ni²⁺ moieties. The particle-immobilized antibodies may then be used to present a protein or other agent which has been fused to the cognate antigen, or be used to directly or indirectly bind to a target agent in a sample solution that is suspected of containing that agent, for example, in a laboratory assay.

“Small molecule,” as used herein, means a molecule less than 5 kilodalton, more typically less than 1 kilodalton. As used herein, “small molecule” excludes proteins.

“Proteins” and “peptides” are well-known terms in the art, and are not precisely defined in the art in terms of the number of amino acids that each includes. As used herein, these terms are given their ordinary meaning in the art. Generally, peptides are amino acid sequences of less than about 50 or about 100 amino acids in length, but can include sequences of up to 300 or 400 amino acids. Proteins generally are considered to be molecules of at least about 100 amino acids.

“Colloid,” as used herein, means nanoparticle, i.e. a very small, self-suspendable particles including inorganic, polymeric, and metal particles. Typically, colloid particles are of less than 250 nm cross section in any dimension, more typically less than 150 or 100 nm cross section in any dimension, and preferably 10-30 nm, and can be metal, non-metal, crystalline or amorphous. As used herein this term includes the definition commonly used in the field of biochemistry.

The term “candidate drug” as used herein, refers to any medicinal substance used in humans, animals, or plants. Encompassed within this definition are compound analogs, naturally occurring, synthetic and recombinant pharmaceuticals, hormones, antimicrobials, neurotransmitters, etc. This includes any substance or precursor (whether naturally occurring, synthetic or recombinant) which is to be evaluated for use as a drug for treatment of neurodegenerative disease, or other disease characterized by aberrant aggregation, or prevention thereof. Evaluation typically takes place through activity in an assay, such as the screening assays of the present invention.

“Fluid suspendable particle” means a particle that can be made to stay in suspension in a fluid in which it is used for purposes of the invention (typically an aqueous solution) by itself, or can be maintained in solution by application of a magnetic field, an electromagnetic field, agitation such as stirring, shaking, vibrating, sonicating, centrifuging, vortexing, or the like. A “magnetically suspendable” particle is one that can be maintained in suspension in a fluid via application of a magnetic field. An electromagnetically-suspendable particle is one that can be maintained in suspension in a fluid by application of an electromagnetic field (e. g., a particle carrying a charge, or a particle modified to carry a charge). A “self-suspendable particle” is a particle that is of low enough size and/or mass that it will remain in suspension in a fluid in which it is used (typically an aqueous solution), without assistance of for example a magnetic field, for at least 1 hour, or for an amount of time that it takes to perform a relevant assay. Other self-suspendable particles will remain in suspension, without assistance, for 5 hours, 1 day, 1 week, or even 1 month, in accordance with the invention.

As used herein, “fastened to or adapted to be fastened,” in the context of a species relative to another species or to a surface of an article, means that the species is chemically or biochemically linked via covalent attachment, attachment via specific biological binding (e. g., biotin/streptavidin), coordinative bonding such as chelate/metal binding, or the like. For example, “fastened” in this context includes multiple chemical linkages, multiple chemical/biological linkages, etc., including, but not limited to, a binding species such as a peptide synthesized on a polystyrene bead, a binding species specifically biologically coupled to an antibody which is bound to a protein such as Protein A, which is covalently attached to a bead, a binding species that forms a part (via genetic engineering) of a molecule such as GST or Phage, which in turn is specifically biologically bound to a binding partner covalently fastened to a surface (e. g., glutathione in the case of GST), etc. As another example, a moiety covalently linked to a thiol is adapted to be fastened to a gold surface since thiols bind gold covalently. Similarly, a species carrying a metal binding tag is adapted to be fastened to a surface that carries a molecule covalently attached to the surface (such as thiol/gold binding) which molecule also presents a chelate coordinating a metal. A species also is adapted to be fastened to a surface if a surface carries a particular nucleotide sequence, and the species includes a complementary nucleotide sequence.

“Covalently fastened” means fastened via nothing other than one or more covalent bonds. For example, a species that is covalently coupled, via EDC/NHS chemistry, to a carboxylate-presenting alkyl thiol which is in turn fastened to a gold surface, is covalently fastened to that surface.

As used herein, a component that is “immobilized relative to” another component either is fastened to the other component or is indirectly fastened to the other component, e. g., by being fastened to a third component to which the other component also is fastened, or otherwise is translationally associated with the other component. For example, a signaling entity is immobilized with respect to a binding species if the signaling entity is fastened to the binding species, is fastened to a colloid particle to which the binding species is fastened, is fastened to a dendrimer or polymer to which the binding species is fastened, etc. A colloid particle is immobilized relative to another colloid particle if a species fastened to the surface of the first colloid particle attaches to an entity, and a species on the surface of the second colloid particle attaches to the same entity, where the entity can be a single entity, a complex entity of multiple species, a cell, another particle, etc. All entities that can be fastened or adapted to be fastened to other entities of the invention also can be immobilized or adapted to be immobilized to the other entities, and vice versa.

“Specifically fastened” or “adapted to be specifically fastened” means a species is chemically or biochemically linked to another specimen or to a surface as described above with respect to the definition of “fastened to or adapted to be fastened,” but excluding all non-specific binding.

“Non-specific binding,” as used herein, is given its ordinary meaning in the field of biochemistry.

“Affinity tag” is given its ordinary meaning in the art. An affinity tag is any biological or chemical material that can readily be attached to a target biological or chemical material. Affinity tags may be attached to a target biological or chemical molecule by any suitable method. For example, in some embodiments, the affinity tag may be attached to the target molecule using genetic methods. For example, the nucleic acid sequence coding the affinity tag may be inserted near a sequence that codes a biological molecule; the sequence may be positioned anywhere within the nucleic acid that enables the affinity tag to be expressed with the biological molecule, for example, within, adjacent to, or nearby. In other embodiments, the affinity tag may also be attached to the target biological or chemical molecule after the molecule has been produced (e.g., expressed or synthesized). As one example, an affinity tag such as biotin may be chemically coupled, for instance covalently, to a target protein or peptide to facilitate the binding of the target to sterptavidin.

Affinity tags include, for example, metal binding tags such as histidine tags, GST (in glutathione/GST binding), streptavidin (in biotin/streptavidin binding). Other affinity tags include Myc or Max in a Myc/Max pair, or polyamino acids, such as polyhistidines. At various locations herein, specific affinity tags are described in connection with binding interactions. The molecule that the affinity tag interacts with (e.g. binds to), which may be a known biological or chemical binding partner, is the “recognition entity.” It is to be understood that the invention involves, in any embodiment employing an affinity tag, a series of individual embodiments each involving selection of any of the affinity tags described herein.

A recognition entity may be any chemical or biological material that is able to bind to an affinity tag. A recognition entity may be, for example, a small molecule such as maltose (which binds to MBP, or maltose binding protein), glutathione, NTA/Ni²⁺, biotin (which may bind to streptavidin), or an antibody. An affinity tag/recognition entity interaction may facilitate attachment of the target molecule, for example, to another biological or chemical material, or to a substrate. Examples of affinity tag/recognition entity interactions include polyhistidine/NTA/Ni²⁺, glutathione S transferase/glutathione, maltose binding protein/maltose, streptavidin/biotin, biotin/streptavidin, antigen (or a fragment of an antigen)/antibody (or a fragment of an antibody), and the like.

As used herein, “chelate coordinating a metal” or metal coordinated by a chelate, refers to a metal coordinated by a chelating agent that does not fill all available coordination sites on the metal, leaving some coordination sites available for binding via a metal binding tag.

As used herein, “metal binding tag/metal/chelate linkage” defines a linkage between first and second species in which a first species is immobilized relative to a metal binding tag and a second species is immobilized relative to a chelate, where the chelate coordinates a metal to which the metal binding tag is also coordinated. U.S. Pat. No. 5,620,850 of Bamdad, et al., incorporated herein by reference, describes exemplary linkages.

A “moiety that can coordinate a metal,” as used herein, means any molecule that can occupy at least two coordination sites on a metal atom, such as a metal binding tag or a chelate.

“Signaling entity” means an entity that is capable of indicating its existence in a particular sample or at a particular location. Signaling entities of the invention can be those that are identifiable by the unaided human eye, those that may be invisible in isolation but may be detectable by the unaided human eye if in sufficient quantity (e. g., colloid particles), entities that absorb or emit electromagnetic radiation at a level or within a wavelength range such that they can be readily detected visibly (unaided or with a microscope including an electron microscope or the like), or spectroscopically, entities that can be detected electronically or electrochemically, such as redox-active molecules exhibiting a characteristic oxidation/reduction pattern upon exposure to appropriate activation energy (“electronic signaling entities”), or the like. Examples include dyes, pigments, electroactive molecules such as redox-active molecules, fluorescent moieties (including, by definition, phosphorescent moieties), up-regulating phosphors, chemiluminescent entities, electrochemiluminescent entities, or enzyme-linked signaling moieties including horse radish peroxidase and alkaline phosphatase.

“Precursors of signaling entities” are entities that by themselves may not have signaling capability but, upon chemical, electrochemical, electrical, magnetic, or physical interaction with another species, become signaling entities. An example includes a chromophore having the ability to emit radiation within a particular, detectable wavelength only upon chemical interaction with another molecule. Precursors of signaling entities are distinguishable from, but are included within the definition of, “signaling entities” as used herein.

As used herein, a “metal binding tag” refers to a group of molecules that can become fastened to a metal that is coordinated by a chelate. Suitable groups of such molecules include amino acid sequences, typically from about 2 to about 10 amino acid residues. These include, but are not limited to, histidines and cysteines (“polyamino acid tags”). Such binding tags, when they include histidine, can be referred to as a “poly-histidine tract” or “histidine tag” or “HIS-tag,” and can be present at either the amino- or carboxy-terminus, or at any exposed region, of a peptide or protein or nucleic acid. A poly-histidine tract of six to ten residues is preferred for use in the invention. The poly-histidine tract is also defined functionally as being a number of consecutive histidine residues added to a protein of interest which allows the affinity purification of the resulting protein on a metal chelate column, or the identification of a protein terminus through the interaction with another molecule (e. g. an antibody reactive with the HIS-tag).

The term “biological binding” refers to the interaction between a corresponding pair of molecules that exhibit mutual affinity or binding capacity, typically specific or non-specific binding or interaction, including biochemical, physiological, and/or pharmaceutical interactions. Biological binding defines a type of interaction that occurs between pairs of molecules including proteins, nucleic acids, glycoproteins, carbohydrates, hormones and the like. Specific examples include antibody/antigen, antibody/hapten, enzyme/substrate, enzyme/inhibitor, enzyme/cofactor, binding protein/substrate, carrier protein/substrate, lectin/carbohydrate, receptor/hormone, receptor/effector, complementary strands of nucleic acid, protein/nucleic acid repressor/inducer, ligand/cell surface receptor, virus/ligand, protein/small molecule, protein/carbohydrate, etc.

The term “determining” refers to quantitative or qualitative analysis of a species via, for example, spectroscopy, ellipsometry, piezoelectric measurement, immunoassay, electrochemical measurement, and the like. “Determining” also means detecting or quantifying interaction between species, e. g. detection of binding between two species.

The term “sample” refers to any cell, tissue, or fluid from a biological source (a “biological sample”), or any other medium, biological or non-biological, that can advantageously be evaluated in accordance with the invention including, but not limited to, a biological sample drawn or derived from a human patient, a sample drawn from an animal, a sample drawn from food designed for human consumption, a sample including food designed for animal consumption such as livestock feed, milk, an organ donation sample, a sample of blood destined for a blood supply, a sample from a water supply, or the like. One example of a sample is a sample drawn from a human or animal to whom a candidate drug has been given to determine the efficacy of the drug.

A “sample suspected of containing” a particular component means a sample with respect to which the content of the component is unknown. For example, a fluid sample from a human suspected of having a disease, such as a neurodegenerative disease or a non-neurodegenerative disease, but not known to have the disease, defines a sample suspected of containing neurodegenerative disease aggregate-forming species. “Sample” in this context includes naturally-occurring samples, such as physiological samples from humans or other animals, samples from food, livestock feed, etc., as well as “structurally predetermined samples,” which are defined herein to mean samples, the chemical or biological sequence or structure of which is a predetermined structure used in an assay designed to test whether the structure is associated with a particular process such as a neurodegenerative disease. For example, a “structurally predetermined sample” includes a peptide sequence, random peptide sequence in a phage display library, and the like. Typical samples taken from humans or other animals include cells, blood, urine, ocular fluid, saliva, cerebro-spinal fluid, fluid or other samples from tonsils, lymph nodes, needle biopsies, etc.

The term “self-assembled monolayer” (SAM) refers to a relatively ordered assembly of molecules spontaneously chemisorbed on a surface, in which the molecules are oriented approximately parallel to each other and roughly perpendicular to the surface. Each of the molecules includes a functional group that adheres to the surface, and a portion that interacts with neighboring molecules in the monolayer to form the relatively ordered array. See Laibinis, P. E., Hickman, J., Wrighton, M. S., Whitesides, G. M., Science, 245:845 (1989); Bain, C., Evall, J., Whitesides, G. M., J. Am. Chem. Soc., 111:7155 (1989); and Bain, C., Whitesides, G. M., J. Am. Chem. Soc., 111:7164-7175 (1989); each of which is incorporated herein by reference.

The present invention makes use of particles with the ability to emit electromagnetic radiation within a narrow wavelength band. The particles may have a diameter of less than 1 or 10 micrometers, preferably less than 100 nanometers, and more preferably less than 10 nanometers. These particles are thus a class of colloid particles. They may include polymeric materials such as polyethylene, loaded or integrated with molecules having the ability to emit fluorescent or ultraviolet radiation in a narrow wavelength band. As used herein, a “narrow wavelength band” is a fluorescent spectrum such that the width of the most intense emission peak at half maximum is narrow enough such that, for example, within the visible spectrum one can determine the existence of at least 5 separate particles simultaneously, preferably at least 8, 10, or even 12 or more particles simultaneously. Preferably, the width of the most intense emission peak at half maximum is about 10 to 50, preferably 20 to 40 nanometers. The fluorescent molecules may be any suitably available fluorescent molecules.

In some embodiments, the particles may comprise a semiconductor nanocrystal.

A semiconductor nanocrystal is a particle of matter, typically with a dimension of less than 100 or 75 nanometers, and more typically less than 50 or 25 nanometers. The radii of a semiconductor nanocrystal may be smaller than the bulk exciton Bohr radius. In a semiconductor nanocrystal, the addition or removal of an electron may change or alter its electronic properties. In certain semiconductor nanocrystals, these electronic properties may include fluorescence, or emission, of light, such as visible light, infrared light, ultraviolet light, or radio frequency radiation.

Semiconductor nanocrystals may be constructed out of any suitable semiconductor material or materials. The semiconductor materials may be, for example, a Group II-VI compound, a Group III-V compound, or a Group IV element. Suitable elements from Group II of the Periodic Table may include zinc, cadmium, or mercury. Suitable elements from Group III may include, for example, gallium or indium. Elements from Group IV that may be used in semiconductor materials may include, for example, silicon, germanium, or lead. Suitable elements from Group V that may be used in semiconductor materials may include, for example, nitrogen, phosphorous, arsenic, or antimony. Appropriate elements from Group VI may include, for example, sulfur, selenium, or tellurium. Examples of suitable Group II-VI compounds include, for example cadmium selenide (CdSe) or cadmium telluride (CdTe). Suitable Group III-V compounds include, for example, gallium arsenide (GaAs) or indium arsenide (InAs). The semiconductor material may include alloys or mixtures of these materials, or different Groups may be combined together, for example, AlGaAs, InGaAs, InGaP, AlGaAs, AlGaAsP, InGaAlP, or InGaAsP.

The emission wavelength of a semiconductor nanocrystal may be governed by the size of the nanocrystal. These emissions may be controlled by varying the particle size or composition of the particle. The light emitted by a semiconductor nanocrystals may have very narrow wavelengths, for example, spanning less than about 100 nm, preferably less than about 80 nm, more preferably less than about 60 nm, more preferably less than about 40 nm, and more preferably less than about 20 nm. The semiconductor nanocrystal may emit a characteristic emission spectrum which can be observed and measured, for example, spectroscopically. Thus, in certain cases, many different semiconductor nanocrystals may be used simultaneously, without significant overlap of the emitted signals. The emission spectra of a semiconductor nanocrystal may be symmetric or nearly so. Unlike some fluorescent molecules, the excitation wavelength of the semiconductor nanocrystal may have a broad range of frequencies. Thus, a single excitation wavelength, for example, a wavelength corresponding to the “blue” region or the “purple” region of the visible spectrum, may be used to simultaneously excite a population of nanocrystals, each of which may have a different emission wavelength. For example, a cadmium selenide crystal of 3 nanometers may produce a 520 nanometer emission, while a cadmium selenide crystal of 5.5 nanometers in diameter may produce a 630 nanometer emission upon excitation with light having a frequency of 450 nanometers, corresponding to “blue” light. Multiple signals, corresponding to, for example, multiple chemical or biological assays, may thus be simultaneously detected and recorded.

In some embodiments of the invention, the quantum dot may further comprise an inner “core” region and an outer “shell” region. The core region may be constructed from a semiconductor material, as previously described above. The shell region may also include a semiconductor material as previously described, or it may include an inorganic or an organic material, such as silicon dioxide (SiO₂), boron, or a polymer, such as latex or polyethylene. The shell may protect the core, amplify the optical properties, insulate the core from the external environment, inhibit photobleaching of the core region, or provide a suitable surface upon which to attach additional chemical functionalities. In certain embodiment of the invention, the semiconductor nanocrystal may be embedded within or attached to a larger structure, for example, a microparticle such as a colloid particle.

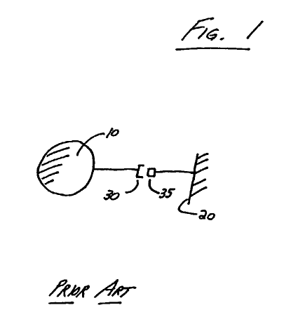

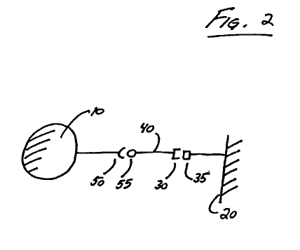

A member of a binding partner pair may be attached to the surface of the particle. A “binding partner,” as used herein, refers to any molecule that can undergo binding with a particular molecule, such as an affinity tag/recognition entity pair. Examples of biological binding partners include Protein A binding with an antibody such as IgG or IgE, or NTA/Ni²⁺ binding with a peptide tag such as a polyhistidine tag. FIG. 1 shows semiconductor nanocrystal 10 bound to surface 20. Semiconductor nanocrystal 10 is bound by the use of binding partners 30, 35, which may represent a covalent linkage. FIG. 2 shows particle 10 of the present invention, attached to surface 20 by two binding partner pair interactions in series. Particle 10 is attached to intermediate entity 40 by a first binding partner pair 50, 55. The intermediate entity, in turn, is bound to surface 20 by a second binding partner pair 30, 35. Either or both of the two sets of binding partner pairs may include an affinity tag/recognition entity interaction pair in this example. In other embodiments, more than two binding partner pair interactions in series may be used to immobilize a member of a binding partner pair with a particle, and some or all of the binding partner pairs may include affinity tag/recognition entity interactions.

In one embodiment, the affinity tag/recognition entity pair is selected from among an antibody/peptide pair, an antibody/antigen pair, an antibody fragment/antigen pair, an antibody/antigen fragment pair, an antibody fragment/antigen fragment pair, an antibody/hapten pair, an enzyme/substrate pair, an enzyme/inhibitor pair, an enzyme/cofactor pair, a protein/substrate pair, a nucleic acid/nucleic acid pair, a protein/nucleic acid pair, a peptide/peptide pair, a protein/protein pair, a small molecule/protein pair, a glutathione/GST pair, a maltose/maltose binding protein pair, a carbohydrate/protein pair, a carbohydrate derivative/protein pair, a metal binding tag/metal/chelate, a peptide/NTA pair, a lectin/carbohydrate pair, a receptor/hormone pair, a receptor/effector pair, a complementary nucleic acid/nucleic acid pair, a ligand/cell surface receptor pair, a virus/ligand pair, a Protein A/antibody pair, a Protein G/antibody pair, a Protein L/antibody pair, an Fc receptor/antibody pair, a biotin/avidin pair, a biotin/streptavidin pair, a drug/target pair, a zinc finger/nucleic acid pair, a small molecule/peptide pair, a small molecule/protein pair, a carbohydrate/protein pair such as maltose/MBP (maltose binding protein), a small molecule/target pair, or a metal ion/chelating agent pair. The affinity tag/recognition entity pair may also be an NTA/Ni²⁺/polyamino acid tag such as polyhistidine, a glutathione/GST pair, an anti-GFP/GFP fusion protein pair, or a Myc/Max pair.

Various ways of immobilizing the binding partner to the semiconductor nanocrystal may be used in the present invention. For example, the semiconductor nanocrystal may be coated with a glass, for example, silicon dioxide, forming the shell region. The member of the affinity tag/recognition entity pair may be affixed to the glass by any suitable means, for example, by covalent bonding or ionic attraction. Alternatively, the semiconductor nanocrystal may be caged or encapsulated in a molecular shell. The molecular shell may be, for example, a polymer, or it may include inorganic materials such as silicon or boron, for example, as a molecular cage. The cage may also be a zeolite in some embodiments. The binding partner may be immobilized on the cage encapsulating the semiconductor nanocrystal by any suitable technique, for example, by covalent attachment.

Additional functionalities may be added to the semiconductor nanocrystals as well. For instance, the semiconductor nanocrystal may carry one or more signals that may be used to identify the attached probe molecule. Examples of such signaling capabilities may include, for instance, characteristics of the particle that are a function of the particle size, optical properties, fluorescent properties of nanoparticle material, fluorescent properties of nanoparticle size, fluorescent properties of attached entities, redox active entities or electroactive entities. Additional or multiple affinity tag/recognition entity partners may also be attached to the semiconductor nanocrystal in some cases. Several semiconductor nanocrystal particles may be used under certain conditions, where the nanocrystal particles may have varying sizes or emission wavelengths.

In some embodiments a member of a binding partner pair (such as a member of an affinity tag/recognition entity pair) may be attached to an intermediate entity. The intermediate entity may be any entity able to become immobilized to the binding partner. For example, the intermediate entity may be a single molecule, such as an organic molecule, a polymer, a protein, a carbohydrate, or a nucleic acid. the intermediate entity may also be a larger entity. For example, the entity might be a colloid particle, a molecular or a protein aggregate, a magnetic particle, a microparticle, a nanoparticle, or a cell.

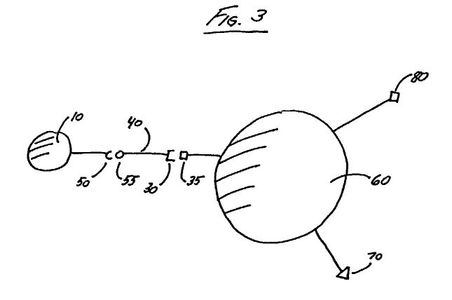

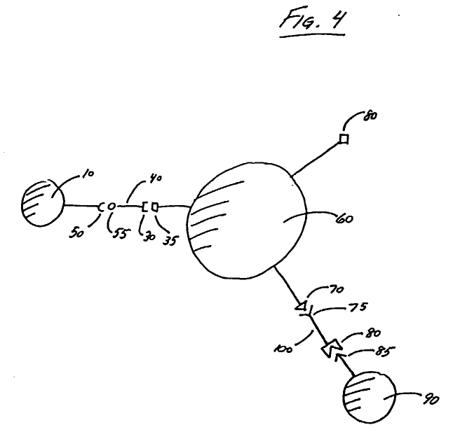

The intermediate entity may have more than one type of binding partner attached to it. For example, in FIG. 3, particle 10 is bound to article 60. Article 60 has several non-identical binding partners 35, 70, 80 attached to its surface. In this embodiment, one of the binding partners 35 is bound to its partner 30 located on intermediate entity 40. Intermediate entity 40 has a second binding partner pair on it 55 bound to its partner 50 located on particle 10. These may be, for instance, affinity tag/recognition entity partners in some embodiments of the invention. However, in other embodiments of the invention, intermediate entity 40 may be absent and particle 10 may be bound to article 60 by only one binding partner pair interaction, which may be an affinity tag/recognition entity interaction. Particle 10 may also be able to emit electromagnetic radiation in a narrow wavelength band as previously described. In FIG. 4, a second particle 90 has also become immobilized to article 60 by one or more binding partner pair interactions. Binding partner 85 is immobilized on particle 90. It is also bound to its partner 80 attached on intermediate entity 100. These may be affinity tag/recognition entity partners in some embodiments of the invention. Intermediate entity 100 further has a second binding partner 75 bound to partner 70 located on article 60. In this embodiment, both particle 10 and particle 90, which may be particles of the same or different sizes and have different detectable properties, such as different wavelengths, may be immobilized to article 60.

In some embodiments of the invention, the particle may be immobilized to a surface via a binding partner pair interaction, such as an affinity tag/recognition entity pair interaction. The surface may be any surface that the member of the binding partner pair may be immobilized upon. For example, the surface may be the surface of a colloid particle, the surface of a semiconductor material, the surface of a magnetic particle, the surface of an electrode, the surface of a fluid suspendable particle, the surface of a magnetically suspendable particle, the surface of an electromagnetically suspendable particle, the surface of a cell, or the surface of a self-suspendable particle. Alternatively, the surface may be the surface of a self-assembled monolayer.

The example arrangement as illustrated in FIG. 2 may occur as follows. Surface 20 of an article may or may not have a chemical or biological agent 35 fastened thereto, and it is a goal to determine whether 35 is or is not fastened to the surface. Particle 10 is prepared with an immobilized binding partner 30 of agent 35, and is immobilized relative to particle 10 via affinity tag/recognition entity pair 50/55. Particle 10 is exposed to surface 20 and, if agent 35 is fastened to surface 20, then particle 10 will become immobilized relative to surface 20 and may be detected. With reference to the example embodiment illustrated in FIG. 3, the presence and/or identity of entities 35, 70, and 80 may be determined by exposing particle 60 to one or more particles 10 carrying the binding partners of entities 35, 70, and 80. In the example embodiment illustrated in FIG. 3 (and FIG. 4), particle 10 and other particles can carry immobilized binding partners 30, etc. directly covalently attached thereto, or attached via affinity tag/recognition entity pair 50/55 (or 70/75 in FIG. 4).

The present invention also provides techniques for drug screening. For example, with reference to FIGS. 2-4, where binding partner pairs 30/35, 70/75, etc. are known to exist and to bind to each other, candidate agents such as drugs for destruction of these interactions (e.g. as in competitive binding) may be introduced into the system and the immobilization of particle 10 relative to surface 20 or article 60 may be indicative of the effectiveness of the candidate drug in disrupting the binding partner interaction.

The function and advantage of these and other embodiments of the present invention will be more fully understood from the examples below. The following examples are intended to illustrate the benefits of the present invention, but do not exemplify the full scope of the invention.

### Prophetic Example 1

### Method for Generating Modular Semiconductor Nanoparticles That Present Universal Acceptor Surfaces

In this example, methods of generating modular semiconductor nanoparticles are described. Various methods have previously been described for the formation of nanoparticles from semiconductor materials such as CdSe. Probe biomolecules can then be attached to these particles without chemical coupling, by binding a recognition entity to the semiconductor nanoparticle that recognizes an affinity tag that has been incorporated into the probe molecule. Detergent-like molecules can also be adsorbed onto the surface of the nanoparticles to render the particles more biocompatible. Molecules with glycol headgroups can be adsorbed onto the nanoparticles to reduce the non-specific binding of irrelevant proteins. Other molecules terminated with nitrilo triacetic acid (NTA) complexed with Ni²⁺ can also adsorbed onto the particles, such that the exposed NTA-Ni²⁺ headgroup captures and presents histidine-tagged proteins which are then used as probes in various assays.

In another embodiment, the particles are coated with detergent-like molecules in a first step. Subsequently, the molecules or polymers derivatized with NTA-Ni²⁺ are attached either covalently or non-covalently to the first surface coating. Histidine-tagged proteins can then be captured and presented by these particles.

### Prophetic Example 2

### Multiplexed Screening of a Sample for the Presence of a Panel of Known Pathogens

This is an example of how a set of nanoparticles, each having a unique, detectable characteristic, can be used with larger beads to perform a multiplexed sandwich assay. A bead, presenting a variety of antibodies against a targeted set of agents, is incubated with a sample suspected of containing an agents. The bed is then exposed to a set of nanoparticles, which each present a single species antibody that recognizes a second site on one of the targeted agents. The nanoparticles each possess a detectable characteristic, which can be used to identify the attached antibody.

Using techniques known to those skilled in the art, antibodies are raised against a panel of targeted agents. For each target agent, a first antibody is selected that binds to a first site on the target and a second antibody is selected that binds to a second site on the target. In this way, these antibody pairs can be used in a variety of sandwich assays.

The panel of first antibodies is attached to a bead or beads (surface). The antibody-presenting bead is then incubated with a sample suspected of containing at least one of these targeted agents. The beads are then washed to remove unrecognized species. Components of the second set of antibodies, that recognize second sites on the individual targets, are then separately attached to a set of nanoparticles that each possess an identifying characteristic, such that each attached antibody can be identified by relating it the detectable characteristic of the attached nanoparticle. This set of antibody-presenting nanoparticles is then incubated with the antigen-bound beads. Unbound nanoparticles are then washed away. The identity of the agents which have been captured by the bead is then determined by detecting the unique characteristics of the captured nanoparticles.

In one embodiment, the antibodies are separately attached to nanoparticles of variable size. By determining the size of the nanoparticle using spectral emissions from the nanoparticle, one can determine which antibody was attached to that particular nanoparticle population. The presence of bead-captured agents is then determined by exposing the nanoparticle-decorated bead to an energy source then reading the spectral emission that defines that set of nanoparticles.

In another embodiment, a set of uniform nanoparticles is derivatized with a set of discrete signaling entities. For example, separate populations of nanoparticles is derivatized with a panel of compounds that fluoresce at different wavelengths. In one embodiment, this is accomplished by incorporating fluorophors attached to thiols into self-assembled monolayers formed on the surfaces of the nanoparticles. The identity of the bead-captured agents is then determined by correlating a signal to a particle to which a known antibody was attached. Alternatively, the nanoparticle-associated signaling entity can be an electroactive entity, including but not limited to redox-active molecules such as ferrocene derivatives.

Examples of samples that can be screened may be derived from sources that include but are not limited to humans (e.g. biologically-derived fluids), animals, food products, water sources, environmental samples, and products of molecular biology and bioengineering.

### Prophetic Example 3

### Using a Set of Signaling Nanoparticles With a Planar Surface

Populations of nanoparticles that each possess a uniquely identifying characteristic can be used to perform multiplexed analysis of species presented on, or captured by, planar surfaces that may or may not be presented in a spatially addressable fashion. For example, using techniques known to those skilled in the art, a surface is prepared such that a variety of biological probes are presented in a spatially addressable format. A sample that contains at least one agent that interacts or is suspected of interacting with a surface-immobilized probe is incubated with the surface. Populations of nanoparticles, each possessing a uniquely identifying characteristic and presenting a single biological probe that interacts with, or is suspected of interacting with, an agent that may be captured by a surface probe, are incubated with the surface. Unbound nanoparticles are then washed away. The identity of the agents captured by the surface is determined by identifying the nanoparticle species bound to each location, for example, using spectrophotometry, and correlating that information with the identity of the attached probe.

Alternatively, a surface can be prepared to non-specifically capture a variety of species that may be present in a sample. The surface is then washed to released unbound species. A collection of nanoparticles that each present both a probe specific for a defined target agent, and a unique, detectable characteristic is then added to the surface. Unbound species are then washed away. The identity of the captured agents is determined by detecting the unique characteristics of the collection of bound nanoparticles, and correlating that information to the set of specific probes that were presented by each nanoparticle species.

In one embodiment, the detectable characteristic is a fluorescent emission that can be correlated to the size of a particular population of nanoparticles to which a specific probe was attached. For example, a surface is coated with Protein A or G, such that it will capture a variety of antibodies by binding to the Fc portion of the antibodies. Antibodies raised against components of Hepatitis B, C, HCV and HIV are incubated with the surface upon which binding takes place, and the antibodies are presented at random locations on the surface. A bodily fluid sample such as blood or serum is added to the surface. Unbound species are then washed away. Four discrete sizes of CdSe or other nanoparticles are separately derivatized to present an antibody that recognizes a different site on one of the target pathogens. Unbound species are then washed from the surface. The surface is subjected to an energy source, for example, blue light, and the emissions are detected and recorded. One is then able to determine whether, for example, the sample tested positive for HIV, by detecting an emission characteristic of the size of the particle to which the HIV antibody had been attached. A surface that uniformly presents a variety of specific antibodies may also be introduced to several samples at distinct spatial locations to facilitate parallel testing of multiple samples.

### Prophetic Example 4

### Multiplexed Detection That Eliminates Wash Steps by Recruiting Nanoparticle-Bound Pathogens to a Sensing Location

A sandwich assay is performed in which a first antibody is attached to a recruitable particle, and a second antibody is attached to a signaling particle. When both particle types simultaneously interact with a target species, a target complex or aggregate, the recruitable particle becomes attached to the signaling particle. The resultant target complex is then recruited to or past a sensing location where a defining characteristic of the nanoparticle is detected. In this example, a first antibody that recognizes a first site on a target species is attached to a magnetic particle. A second antibody that recognizes a second site on the target species is attached to a nanoparticle that fluoresces at a particular wavelength determined by the size of the particle. Different antibodies that recognize different targets are separately immobilized on a panel of variable size nanoparticles, such that each discrete size of nanoparticle can be correlated to a particular antibody that was attached to that size particle. Populations of both magnetic and signaling particles are pooled then incubated with a sample suspected of containing the target agent. Binding is allowed to occur. The resultant multi-particle complex is then electromagnetically drawn to or through a sensing location where the size of the nanoparticle, and thus the identity of the target agent, is determined by detecting spectral emissions.

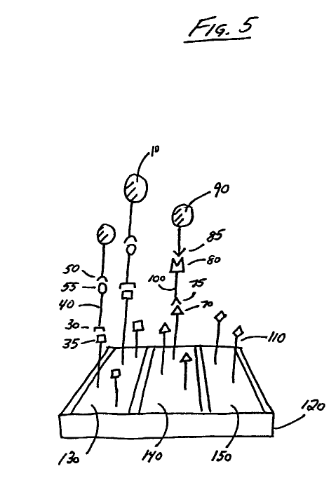

In FIG. 5, binding partners 35, 70 may be two different types of antibodies. These binding partners may bind to their respective targets 40, 70 through binding partners 30, 75, respectively. The targets may be, for example, proteins, such as proteins obtained from a cDNA library or proteins from a cell extract. Targets 40, 70 may become attached to particles 10, 90 through a binding partner, such as affinity tag/recognition entity pairings 50, 55 and 80, 85. Particles 10, 90 may emit electromagnetic radiation, for example as in a semiconductor nanocrystals.

Alternatively, the strategy described above can be carried out to identify binding partners from pools of putative binding pairs. In this case, a first set of putative binding partners is attached to a first set of magnetic particles and a second set of candidate binders are separately attached to a second set of signaling nanoparticles. Binding is then allowed to occur. The resultant complexes are magnetically drawn to a sensing location where the optical properties of the recruited nanoparticles are detected. In a preferred embodiment, energy is added and fluorescent emissions are sensed.

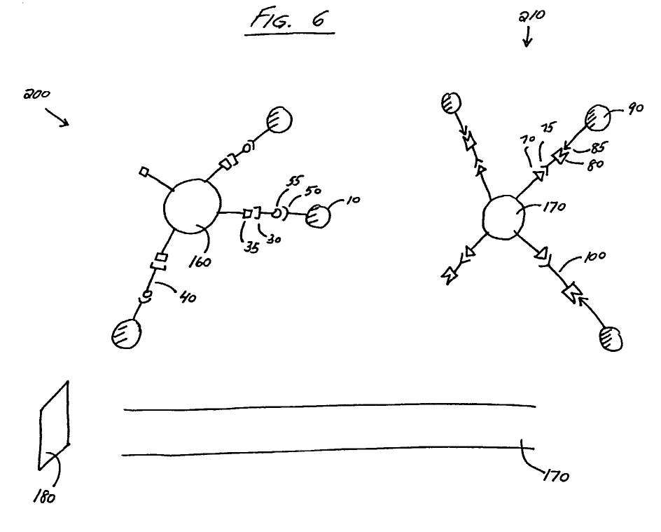

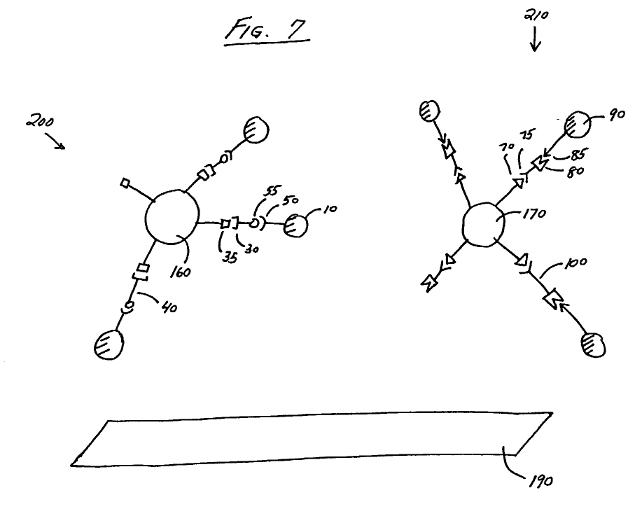

Proteins can be obtained from cDNA libraries. In FIG. 6, two articles 160, 170 each have several particles 10, 90 immobilized to them via two binding partner pair interactions in series 30/35, 50/55, 70/75, and 80/85, forming complexes 200 and 210, respectively. The articles may be, for example, fluid-suspendable particles. Complexes 200, 210 may pass through a beam 170 to be detected by detector 180. For example, beam 170 may be a beam of “blue” or “purple” light and detector 180 may be a photomultiplier tube or a diffraction grating. Alternatively, in FIG. 7, articles 160, 170 may be magnetic beads, which may be drawn to an electrode 190. The beads may be drawn to the electrode magnetically, or by any other suitable technique. Electrode 190 may detect electrical properties of the complexes 200, 210, or may detect emission spectra or other physical or chemical characteristics of the complexes 200, 210.

While several embodiments of the invention have been described and illustrated herein, those of ordinary skill in the art will readily envision a variety of other means and structures for performing the functions and/or obtaining the results or advantages described herein, and each of such variations or modifications is deemed to be within the scope of the present invention. More generally, those skilled in the art would readily appreciate that all parameters, dimensions, materials, and configurations described herein are meant to be exemplary and that actual parameters, dimensions, materials, and configurations will depend upon specific applications for which the teachings of the present invention are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described. The present invention is directed to each individual feature, system, material and/or method described herein. In addition, any combination of two or more such features, systems, materials and/or methods, if such features, systems, materials and/or methods are not mutually inconsistent, is included within the scope of the present invention.

In the claims (as well as in the specification above), all transitional phrases such as “comprising,” “including,” “carrying,” “having,” “containing,” “involving,” and the like are to be understood to be open-ended, i.e. to mean including but not limited to. Only the transitional phrases “consisting of” and “consisting essentially of” shall be closed or semi-closed transitional phrases, respectively, as set forth in the United States Patent Office Manual of Patent Examining Procedures, section 2111.03.

What is claimed is:

1. A complex, comprising:
a species able to emit electromagnetic radiation in a narrow wavelength band; and
a member of an affinity tag/recognition entity pair immobilized relative to the species.

2. The complex of claim 1, further comprising a particle immobilized relative to the species.

3. The complex of claim 2, wherein the particle is a colloid particle.

4. The complex of claim 2, wherein the particle is fluid-suspendable.

5. The complex of claim 2, wherein the species is positioned internally of the particle.

6. The complex of claim 2, wherein the species is fastened to the particle.

7. The complex of claim 2, wherein the particle is immobilized relative to the species via a binding partner pair interaction.

8. The complex of claim 1, wherein the species comprises a semiconductor nanocrystal.

9. The complex of claim I, wherein the narrow wavelength band is affected by a dimension of the species.

10. The complex of claim 1, wherein the species has a largest dimension of less than about 10 micrometers.

11. The complex of claim 1, wherein the species has a largest dimension of less than about 250 nm.

12. The complex of claim 1, wherein the species has a largest dimension of less than about 100 nm.

13. The complex of claim 1, wherein the species has a largest dimension of less than about 30 nm.

14. The complex of claim 1, wherein the species has a largest dimension of less than about 10 nm.

15. The complex of claim 2, wherein the particle has a largest dimension of less than about 10 micrometers.

16. The complex of claim 2, wherein the particle has a largest dimension of less than about 250 nm.

17. The complex of claim 2, wherein the particle has a largest dimension of less than about 100 nm.

18. The complex of claim 2, wherein the particle has a largest dimension of less than about 30 nm.

19. The complex of claim 2, wherein the particle has a largest dimension of less than about 10 nm.

20. The complex of claim 1, wherein the member of the affinity tag/recognition entity pair is selected from the group consisting of:
an antibody/peptide pair,
an antibody/antigen pair,
a fragment of an antibody/antigen pair,
a nucleic acid/nucleic acid pair,
a protein/nucleic acid pair,
a peptide/peptide pair,
a protein/protein pair,
a small molecule/protein pair,
a glutathione/GST pair,
a maltose/maltose binding protein pair,
a carbohydrate/protein pair,
a carbohydrate derivative/protein pair,
a peptide tag/metal ion-metal chelate pair,
a peptide/NTA-Ni pair,
a Protein A/antibody pair,
a Protein G/antibody pair,
a Protein L/antibody pair,
an Fc receptor/antibody pair,
a biotin/avidin pair,
a biotin/streptavidin pair,
a zinc finger/nucleic acid pair,
a small molecule/peptide pair,
a small molecule/target pair, and
a metal ion/chelating agent/polyamino acid pair.

21. The complex of claim 1, wherein the member of the affinity tag/recognition entity pair comprises a polyamino acid sequence.

22. The complex of claim 21, wherein the polyamino acid sequence comprises a polyhistidine sequence.

23. The complex of claim 1, wherein the member of the affinity tag/recognition entity pair is selected from the group consisting of a NTA-Ni²⁺/histidine pair.

24. The complex of claim 1, wherein the member of the affinity tag/recognition entity pair is selected from the group consisting of a glutathione/GST pair.

25. The complex of claim 1, wherein the member of the affinity tag/recognition entity pair is selected from the group consisting of an anti-GFP/GFP fusion protein pair.

26. The complex of claim 1, wherein the member of the affinity tag/recognition entity pair is selected from the group consisting of a Myc/Max pair.

27. The complex of claim 1, wherein the electromagnetic radiation comprises visible radiation.

28. The complex of claim 1, wherein the electromagnetic radiation comprises infrared radiation.

29. The complex of claim 1, wherein the electromagnetic radiation comprises ultraviolet radiation.

30. The complex of claim 1, wherein the electromagnetic radiation comprises radiofrequency radiation.

31. The complex of claim 1, wherein the narrow wavelength band has a width at half maximum of less than about 50 nm.

32. The complex of claim 1, wherein the narrow wavelength band has a width at half maximum of less than about 40 nm.

33. The complex of claim 2, wherein the member of the affinity tag/recognition entity pair is fastened to the particle.

34. The complex of claim 2, wherein the member of the affinity tag/recognition entity pair is immobilized relative to the particle via a binding partner pair interaction.

35. A method, comprising:
providing a complex comprising a particle and a chemical or biological entity, the complex able to emit electromagnetic radiation in a narrow wavelength band; and
exposing the complex to a fluid suspected of comprising a substance able to bind to the chemical or biological entity.

36. A complex, comprising:
an article; and
a species able to emit electromagnetic radiation in a narrow wavelength band immobilized relative to the article via at least two binding partner pair interactions in series.

37. The complex of claim 36, further comprising a particle that the species is immobilized relative to.

38. The complex of claim 37, wherein the species is positioned internally of the particle.

39. The complex of claim 37, wherein the species is fastened to the particle.

40. The complex of claim 36, wherein the species comprises a semiconductor nanocrystal.

41. The complex of claim 36, wherein the narrow wavelength band is affected by a dimension of the species.

42. The complex of claim 36, wherein at least one of the at least two binding partner pair interactions comprises an affinity tag/recognition entity interaction.

43. The complex of claim 42, wherein the affinity tag/recognition entity interaction comprises an interaction selected from the group consisting of:
an antibody/peptide interaction,
an antibody/antigen interaction,
a fragment of an antibody/antigen interaction,
a nucleic acid/nucleic acid interaction,
a protein/nucleic acid interaction,
a peptide/peptide interaction,
a protein/protein interaction,
a small molecule/protein interaction,
a glutathione/GST interaction,
a maltose/maltose binding protein interaction,
a carbohydrate/protein interaction,
a carbohydrate derivative protein interaction,
a peptide tag/metal ion-metal chelate interaction,
a peptide/NTA-Ni interaction,
a Protein A/antibody interaction,
a Protein G/antibody interaction,
a Protein L/antibody interaction,
an Fc receptor/antibody interaction,
a biotin/avidin interaction,
a biotin/streptavidin interaction,
a zinc finger/nucleic acid interaction,
a small molecule/peptide interaction,
a small molecule/target interaction, and
a metal ion/chelating agent/polyamino acid interaction.

44. The complex of claim 42, wherein the affinity tag/recognition entity interaction comprises an interaction with a polyamino acid sequence.

45. The complex of claim 44, wherein the polyamino acid sequence comprises a polyhistidine sequence.

46. The complex of claim 42, wherein the affinity tag/recognition entity interaction comprises a NTA-Ni²⁺/histidine tag interaction.

47. The complex of claim 42, wherein the affinity tag/recognition entity interaction comprises a glutathione/GST interaction.

48. The complex of claim 42, wherein the affinity tag/recognition entity interaction comprises an anti-GFP/GFP fusion protein interaction.

49. The complex of claim 42, wherein the affinity tag/recognition entity interaction comprises a Myc/Max pair.

50. The complex of claim 36, wherein the article comprises a particle.

51. The complex of claim 36, wherein the article comprises a colloid particle.

52. The complex of claim 36, wherein the article comprises a semiconductor material.

53. The complex of claim 36, wherein the article comprises a self-assembled monolayer.

54. The complex of claim 36, wherein the article comprises a magnetic particle.

55. The complex of claim 36, wherein the article comprises an electrode.

56. The complex of claim 36, wherein the article comprises a cell.

57. The complex of claim 36, wherein the article comprises a chip.

58. The complex of claim 36, wherein the electromagnetic radiation comprises visible radiation.

59. A method, comprising:
providing a complex having at least two binding partner pair interactions in series, the complex able to emit electromagnetic radiation in a narrow wavelength band; and
exposing the complex to a fluid suspected of comprising a substance able to bind to at least one of the at least two binding partner pair interactions.

60. A complex, comprising:
an article comprising a particle comprising a first binding partner and a second binding partner not identical to the first binding partner; and
a species able to emit electromagnetic radiation in a narrow wavelength band immobilized relative to the article.

61. The complex of claim 60, wherein the particle further comprises a third binding partner not identical to the first or second binding partners.

62. The complex of claim 61, wherein the particle further comprises a fourth binding partner not identical to the first, second, or third binding partners.

63. The complex of claim 62, wherein the particle further comprises a fifth binding partner not identical to the first, second, third, or fourth binding partners.

64. The complex of claim 60, wherein the species is fastened to the article.

65. The complex of claim 60, wherein the species is immobilized relative to the particle.

66. The complex of claim 60, wherein the species is positioned internally of the article.

67. The complex of claim 60, wherein the species is fastened to the article

68. The complex of claim 60, wherein the species comprises a semiconductor nanocrystal.

69. The complex of claim 60, wherein the narrow wavelength band is affected by a dimension of the species.

70. The complex of claim 60, wherein the first binding partner comprises a member of an affinity tag/recognition entity pair.

71. The complex of claim 60, wherein the first binding partner is immobilized relative to the article via an affinity tag/recognition entity interaction.

72. The complex of claim 70, wherein the member of the affinity tag/recognition entity pair comprises a polyamino acid sequence.

73. The complex of claim 72, wherein the polyamino acid sequence comprises a polyhistidine sequence.

74. The complex of claim 70, wherein the member of the affinity tag/recognition entity pair is selected from the group consisting of a NTA-Ni²⁺/histidine tag pair.

75. The complex of claim 70, wherein the member of the affinity tag/recognition entity pair is selected from the group consisting of a glutathione/GST pair.

76. The complex of claim 70, wherein the member of the affinity tag/recognition entity pair is selected from the group consisting of an anti-GFP/GFP fusion protein pair.

77. The complex of claim 70, wherein the member of the affinity tag/recognition entity pair is selected from the group consisting of a Myc/Max pair.

78. The complex of claim 60, wherein the particle is a colloid particle.

79. The complex of claim 60, wherein the electromagnetic radiation comprises visible radiation.

80. The complex of claim 60, wherein the narrow wavelength band has a width at half maximum of less than about 50 nm.

81. A method, comprising:
providing a complex able to become immobilized relative to an article comprising at least a first binding partner and a second binding partner not identical to the first binding partner, the complex able to emit electromagnetic radiation in a narrow wavelength band; and
exposing the complex to a fluid suspected of comprising a substance able to alter the ability of complex particle to become immobilized relative to the article.

82. An system, comprising:
an article;
a first particle immobilized relative to the article via a first binding partner pair interaction, the first particle comprising a first species able to emit electromagnetic radiation in a first narrow wavelength band; and
a second particle immobilized relative to the article via a second binding partner pair interaction, the second particle comprising a second species able to emit electromagnetic radiation in a second narrow wavelength band.

83. The system of claim 82, wherein the first particle is a colloid particle.

84. The system of claim 82, wherein the first species is positioned internally of the first particle.

85. The system of claim 82, wherein the first species is fastened to the particle.

86. The system of claim 82, wherein the first narrow wavelength band is affected by a dimension of the first species.

87. The system of claim 82, wherein the article comprises a particle.

88. A method, comprising:
allowing a first particle to become immobilized relative to an article via a first binding partner pair interaction, the first particle comprising a first species able to emit electromagnetic radiation in a first narrow wavelength band; and
allowing a second particle to become immobilized relative to the article via a second binding partner pair interaction, the second particle comprising a second species able to emit electromagnetic radiation in a second narrow wavelength band.
